(19) United States
(12) Patent Application Publication
(10) Pub. No.: US 2024/0269677 A1
(43) Pub. Date: Aug. 15, 2024

(54) HERMETICALLY SEALABLE CONTAINER FOR LIQUIDS

(71) Applicant: VACUTEST KIMA S.R.L., PADOVA (Arzergrande) (IT)

(72) Inventors: Renzo CHIARIN, PADOVA (Arzergrande) (IT); Ulisse CHIARIN, Arzergrande (Padova) (IT)

(21) Appl. No.: 18/563,779

(22) PCT Filed: May 20, 2022
(86) PCT No.: PCT/IB2022/054732

§ 371 Date: Nov. 22, 2023

(30) Foreign Application Priority Data
May 26, 2021 (IT) 102021000013640

(51) Int. Cl. B01L 3/00

(52) U.S. Cl. CPC B01L 3/50825; B01L 2200/0689; B01L 2200/082; B01L 2200/141; B01L 2300/042

(57) ABSTRACT

A hermetically sealable container for liquids has a container body and a cap for closing a mouth of the container body. The cap has a main gripping body and a coupling appendage for insertion in the container body. An inner surface of a tubular container wall has a conical guide surface and an intermediate surface connecting the conical guide surface to a shoulder and defining a first annular prominence. The coupling appendage may be in contact with a head end against the shoulder to create a first sealing zone. When the head end abuts against the shoulder, the head end interferes with the first annular prominence, creating a second sealing zone. The coupling appendage has a second annular prominence protruding from a lateral coupling surface near a base of the coupling appendage. When the head end abuts against the shoulder, the second annular prominence interferes with the conical guide surface, creating a third sealing zone.

### FIELD OF APPLICATION

The present invention refers to a hermetically sealable container for liquids.

The hermetically sealable container for liquids according to the invention may be used for the containment of any liquid.

Specifically, the container according to the invention is intended for the containment of liquids for which the risk of conveying the contents from the container to the external environment is to be reduced by means of the sealing cap.

Still more specifically, the container according to the invention is intended for the containment of contaminating and/or hazardous liquids, such as biological samples. In this case, the container may be made, in particular, in the form of a jar or test tube.

### PRIOR ART

A hermetically sealable container for liquids comprises a container body, which delimits the volume of the container, and a cap, which is configured to close the mouth of the container body and make a hermetic seal therewith to prevent the contents from escaping.

Generally, a hermetic seal is achieved by interposing a gasket made of elastomeric material between the container body and the cap.

An alternative solution involves equipping the cap with an appendage made of elastic polymer material, intended to be inserted inside the container body. The appendage is dimensioned to couple by interference with the inner walls of the container and by deforming, thus forming a hermetic seal. This solution is adopted especially for vacuum-sealed tubes.

There are also containers in which the caps do not have appendages made of polymeric material, but in which the cap and container body are both made entirely of plastics material and the hermetic seal is achieved without the use of elastomeric gaskets. These containers are more economical to make. The cap may be coupled to the container by screwing or by simple pressure.

In the containers shown, for example, in FIGS. 1 to 4 (jar and swab tube), the cap C is screwed onto the container body B, and for this purpose the container body has a threaded portion F formed on the outer surface near the mouth. The cap C further comprises an inner appendage D intended to fit inside the mouth of the container body. Said inner appendage D is slightly conical so as to allow easy insertion of said appendage into the mouth and is dimensioned so that, past a certain level of screwing of the cap onto the container body, the appendage begins to interfere with the inner walls of the container body. A hermetic seal is achieved by forcing the cap by screwing until it comes to rest on the edge of the mouth.

Because of its conicity, the inner appendage D does not adhere to the inner walls of the container body along its entire length, but is spaced therefrom at least at its head portion. An annular cavity E is then formed between the appendage D and the container body B, which cavity, due to capillary phenomena, becomes an area wherein some of the liquid contents of the container may easily stagnate and accumulate. In fact, it is not uncommon for a container to be shaken or tilted enough during transport or handling to cause the contents to lap against the cap. Upon opening the container, the liquid accumulated in the aforesaid annular cavity E is pulled away by the cap and may accidentally leak into the environment in which the container is handled.

The same issue arises if the plastics cap is configured to engage the container body under pressure, as in the example of the container (test tube) shown in FIGS. 5 and 6. Also in this case, in fact, the cap C is equipped with an inner appendage D that is intended to fit inside the container body B and that inevitably forms an annular cavity E because of the necessary conicity with which the head portion of the inner appendage D is endowed.

This issue is particularly acute when containers are intended to contain potentially contaminating and/or hazardous liquids. In the case of biological samples that are potentially contaminated with pathogens, this increases the risk of contamination of the environments and/or operators handling said samples.

Although operators assigned to handle containers containing biological specimens are aware of these risks and are therefore able to put in place procedures to minimize them, a need is felt in the relevant sector for hermetically sealable containers that are configured to inherently reduce the risk of contamination due to the conveyance of the biological specimen to the external environment via the container lid.

Conveying the contents outside the container via the cap may be problematic even if this does not involve biological and/or hazardous liquids. One may consider the case of liquids that may soil or stain.

Thus, the need for hermetically sealable containers that are configured to inherently reduce the risk of contamination due to the conveyance of contents to the external environment via the container cap cuts across many areas of application.

To date, however, this need is completely unsatisfied as no hermetically sealable containers for liquids, which are made entirely of plastics and which do not have the drawbacks described above, are available.

### DISCLOSURE OF THE INVENTION

Thus, the main object of the present invention is to eliminate or at least mitigate the drawbacks of the aforesaid prior art by providing a hermetically sealable container for liquids that, while being completely made of plastics, allows for the significant reduction of the risk of contamination by conveying the contents to the external environment via said container cap.

A further object of the present invention is to provide a hermetically sealable container for liquids that ensures a hermetic seal comparable to similar containers of known types.

A further object of the present invention is to provide a hermetically sealable container for liquids which is simple and inexpensive to manufacture.

### BRIEF DESCRIPTION OF THE DRAWINGS

The technical features of the invention, according to the aforesaid objects, may be clearly seen in the contents of the claims below, and its advantages will become more readily apparent in the detailed description that follows, made with reference to the accompanying drawings, which represent one or more purely exemplifying and non-limiting embodiments thereof, wherein:

FIG. 1 is an orthogonal elevation view in cross section of an example of a hermetically sealable container for liquids of a known type, consisting of a biological specimen jar fitted with a screw-on cap;

FIG. 2 is an enlarged detail of the container of FIG. 1;

FIG. 3 is an orthogonal elevation view in cross section of a further example of a known type of a hermetically sealable container for liquids, consisting of a swab tube equipped with a screw-on cap;

FIG. 4 is an enlarged detail of the container of FIG. 3;

FIG. 5 is an orthogonal elevation view in cross section of a further example of a known type of a hermetically sealable container for liquids, consisting of a test tube fitted with a pressure cap;

FIG. 6 is an enlarged detail of the container of FIG. 1;

FIG. 7 shows an orthogonal elevation view in cross section of a hermetically sealable container for liquids according to a first embodiment of the invention, consisting of a jar for biological samples fitted with a screw-on cap;

FIG. 8 is an enlarged detail of the container of FIG. 7;

FIG. 9 is an enlarged detail of the cap of the container in FIG. 8 separated from the container body;

FIG. 10 is an enlarged detail of the container body of the container of FIG. 8 separated from the cap;

FIG. 11 is an orthogonal elevation view in cross section of a hermetically sealable container for liquids according to a second embodiment of the invention, comprised of a swab tube fitted with a screw-on cap;

FIG. 12 is an enlarged detail of the container of FIG. 11;

FIG. 13 is a further enlarged detail of the container in FIG. 12;

FIG. 14 is an enlarged detail of the container cap in FIG. 13 separated from the container body;

FIG. 15 is an enlarged detail of the container body of the container in FIG. 13 separated from the cap;

FIG. 16 is an orthogonal elevation view in cross section of a hermetically sealable container for liquids according to a third embodiment of the invention, consisting of a test tube fitted with a pressure cap;

FIG. 17 is an enlarged detail of the container in FIG. 16;

FIG. 18 is an enlarged detail of the container body of the container of FIG. 17 separated from the cap; and

FIG. 19 is an enlarged detail of the container cap in FIG. 17 separated from the container body.

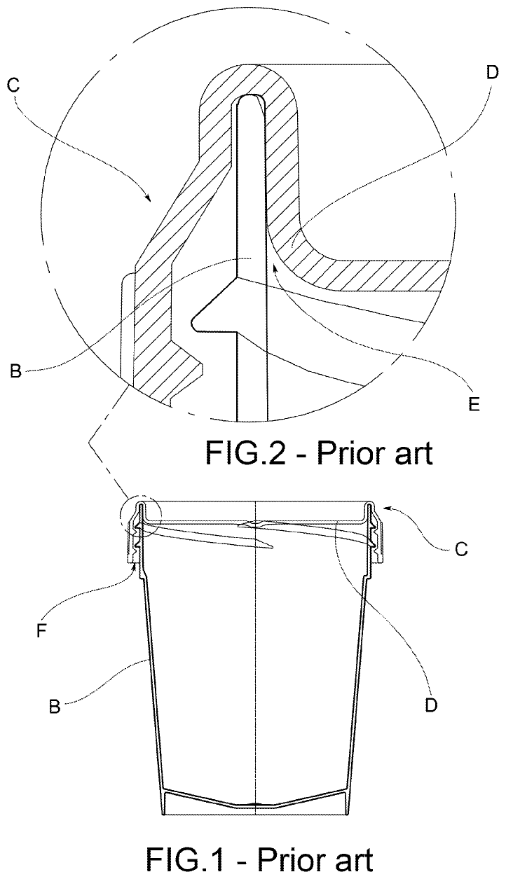

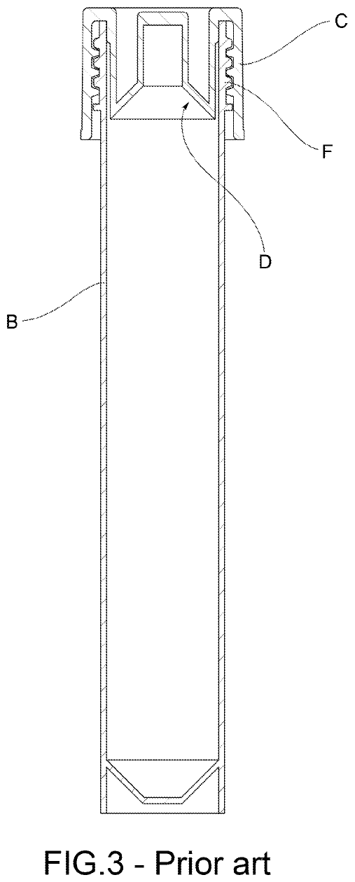

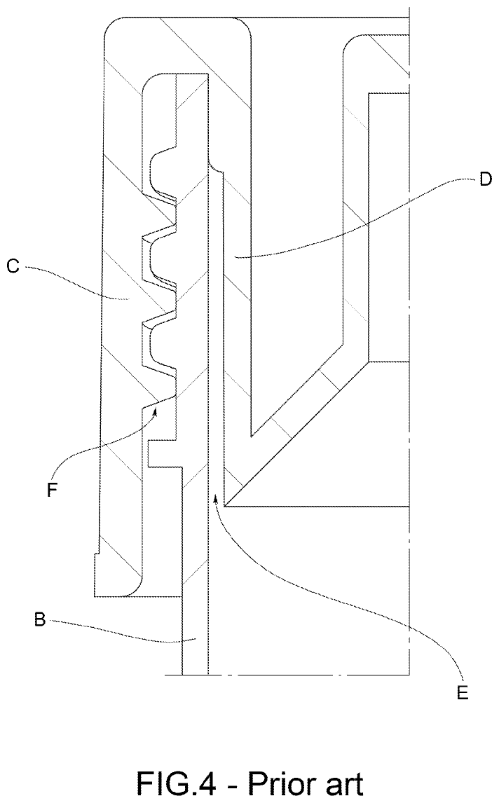

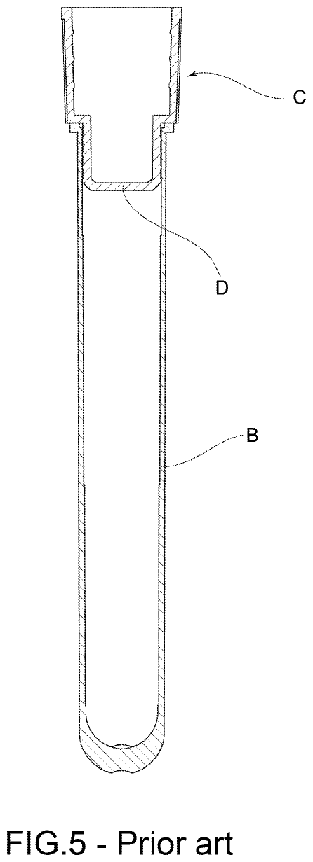

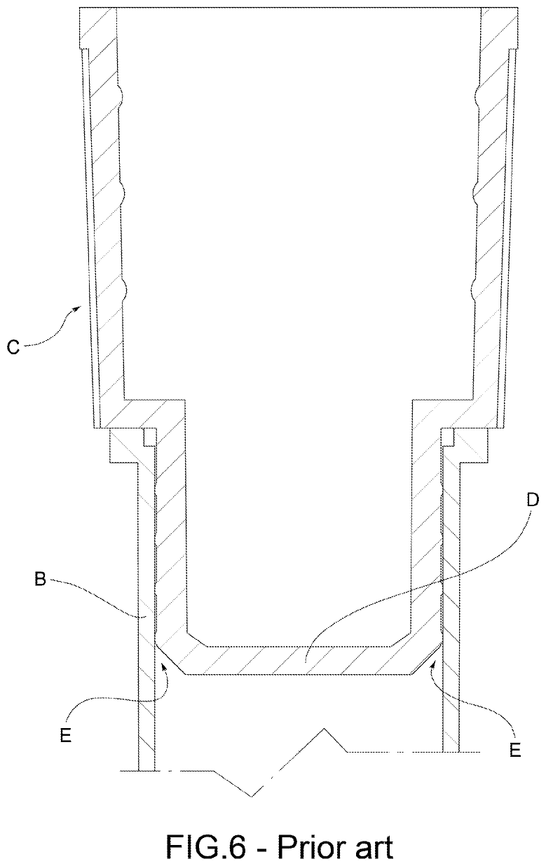

### DETAILED DESCRIPTION

The hermetically sealable container for liquids according to the invention has been denoted collectively with 1 in the attached figures.

Herein and in the following description and the claims, reference will be made to the container 1 in the condition of use. Therefore, any references to a lower or upper position or to a horizontal or vertical orientation should be interpreted in this sense.

According to a general embodiment of the invention, the hermetically sealable container 1 for liquids comprises a container body 10, which delimits the inner volume of the container laterally by a tubular container wall 11 and at the bottom by a container bottom 12, from which said tubular container wall 11 extends. The container body 10 has a mouth 13 delimited by a free edge 14 of said tubular wall 11.

An inner surface area 110 and an outer surface area 120 may be identified on the tubular body 11.

The hermetically sealable liquid container 1 further comprises a cap 20, configured to close the mouth 13 of container body 10 and form a hermetic seal therewith.

In turn, the cap 20 comprises a main gripping body 21, which is intended to remain external to the container body 10 and through which the cap is manipulable by a user.

The cap 20 further comprises a coupling appendage 22, which:
is intended to be inserted inside the container body 10 through the mouth 13,
extends axially between a base 220 and a head end 221, and
comprises a lateral coupling surface 23 intended to face the inner surface 110 of said tubular container part 11.

The container body 10 and the cap 20 are made completely of plastics.

Preferably, both the container body 10 and the cap 20 are made by injection molding of plastics material.

Preferably, the container body 10 is made of a plastics material chosen from the group consisting of polypropylene, polyethylene, polystyrene, PET, PVC, polycarbonate, methacrylate, and the copolymers thereof.

Preferably, the cap 20 is made of a plastics material chosen from the group consisting of polypropylene, polyethylene, polystyrene, PET, PVC, polycarbonate, methacrylate, and the copolymers thereof.

According to a first aspect of the invention, the inner surface 110 of said tubular container wall 11 comprises sequentially from the free edge 14 toward the bottom 12:
a conical guide surface 111, converging toward the bottom 12;
an intermediate surface 112, connecting said conical guide surface 111 to a shoulder 113;
an end surface 114 connecting said shoulder 113 to the container bottom 12.

Functionally, the conical guide surface 111 is suitable to facilitate the insertion of the coupling appendage 22 of the cap 20 inside the container body 10 through the mouth 13. For this purpose, the conical guide surface 111 has a minimum diameter greater than the maximum diameter of the coupling appendage 22, with the exclusion of specific portions of the coupling appendage 22, which, as will be taken up in the following, are specifically dimensioned to engage in an interference relationship on the conical guide surface 111.

Advantageously, the aforesaid conical guide surface 111 has a conicity between 1/1 and 1/50.

The conicity C is a dimensionless quantity that expresses the ratio between the difference of the diameters D and d of two cross sections of a cone and the axial distance L between them, expressed by the relationship C=(D−d)/L. For example, a conicity C equal to 1:20 means that, for an axial distance of 20 millimeters between two cross sections, the major diameter D and minor diameter d will differ by 1 millimeter.

Functionally, the intermediate surface 112 is suitable to define a connection surface between the conical guide surface 111 and the shoulder 113.

The aforesaid intermediate surface 112 may be conical converging toward the bottom 12 or cylindrical.

Preferably, if the intermediate surface 112 is conical, it has a conicity lower than the conicity of the conical guide surface 111.

The aforesaid intermediate surface 112 defines a first annular prominence 115 in the vicinity of said shoulder 113. Functionally, said first annular prominence 115 is suitable to define a localized bottleneck section of the tubular container wall 11 at the inner surface 110.

According to a second aspect of the invention, the coupling appendage 22 of said cap 20 has an axial extension such that the coupling appendage 22 may be inserted within the container body 11 until it reaches said shoulder 113 with the head end 221. In other words, the coupling appendage 20 has an axial development (distance between the base 220 and the head end 221) at least equal to the distance between the free edge 14 and the shoulder 113.

The coupling appendage 22 of said cap 20 also has a radial extension (in a plane cross-sectional to the axis) at the head end 221 such that, when it reaches the shoulder 113, the head end 221 abuts against said shoulder 113, so as to create a first sealing zone T1 between the cap (20) and the container body 10.

According to a third aspect of the invention, the head end portion 221 of the coupling appendage 22 is radially dimensioned such that when the head end 221 abuts against the shoulder 113, the head end portion 221 interferes with the aforesaid first annular prominence 115, creating a second sealing zone T2 between the cap 20 and the container body 10.

According to a fourth aspect of the invention, the coupling appendage 22 of said cap (20) comprises a second annular prominence 222 which protrudes from said lateral coupling surface 23 in the vicinity of the base 220 of said coupling appendage 22 and is positioned axially such that, when the head end 221 abuts against said shoulder 113, the second annular prominence 222 interferes with the conical guide surface 111, creating a third sealing zone T3 between the cap 20 and the container body 10.

Due to the invention, the coupling appendage 22 adheres to the inner surface 110 of the tubular wall 11 of the container body 10 at least at its own head end 221. More specifically, by creating an initial sealing zone T1 at the head end 221, the seepage of the container contents between the coupling appendage 22 and the tubular wall 11 is prevented. Thus, an open annular cavity is not formed between the coupling appendage 22 and the container body 10 on the inside of said container. All this prevents the contents of the container from seeping between the coupling appendage and the container body and stagnating in said position. Therefore, when the container is opened, as there is no liquid accumulated between the appendage and the container body, the risk of the contents being conveyed outside the container via said cap is significantly reduced.

This result is achieved without making the coupling appendage 22 of elastomeric material or even endowing this appendage with seals made of elastomeric material.

The insertion of the coupling appendage inside the container body is facilitated by the conical guide surface 111 formed near the free edge 14 of the container body. This allows for easy insertion inside the container body without making the head end portion of the coupling appendage conical. The absence of conicity on the head end portion 221 of the coupling appendage synergistically helps to avoid the creation of annular cavities (i.e., areas of potential liquid stagnation) between the coupling appendage and tubular wall.

The seepage of liquid between the coupling appendage and the tubular wall is also counteracted by the second sealing zone T2 made between the tubular wall 11 and the head end portion 221 near the shoulder 113.

Preferably, precisely to enhance the effectiveness of the aforesaid second sealing zone T2, the intermediate surface 112 defines the first annular prominence 115 against said shoulder 113. In other words, the first annular prominence 115 connects directly with the shoulder 113. Thus, the second sealing zone T2 is seamlessly connected to the first sealing zone T1.

Functionally, the seal generated at the first sealing zone T1 results from the axial compression of the coupling appendage 22 against the tubular wall 11 at the shoulder 113. Conversely, the seal generated at the second sealing zone T2 results from the radial compression of the coupling appendage 22 against the tubular wall 11 at the first annular prominence 115.

The third sealing zone T3 between the coupling appendage 22 and the tubular wall 11, due to the interference between the conical guide surface 111 and second annular prominence 222, has a dual function:
improving the hermetic seal of the cap 22 on the container body; and
keeping the coupling appendage 22 axially centered within the tubular wall 11 so as to avoid radial movements of the cap relative to the container body; said movements could in fact negatively interfere with the sealing effectiveness of the first and second sealing zones.

From the foregoing, it clearly appears that the hermetically sealable container 1 for liquids according to the invention, while being completely made of plastics, allows for a significant reduction in the risk of contamination by conveying the contents to the external environment via the container cap.

Further, the container 1 according to the invention provides a hermetic seal comparable to similar containers of a known type due to the presence of as many as three sealing zones T1, T2 and T3.

Advantageously, the coupling surface 23 of the coupling appendage 22 is substantially cylindrical, except for the portion on which the second annular prominence 222 is formed.

The term “substantially cylindrical” is also intended to include cases in which the coupling appendage has a coupling surface 23 endowed with a slight or very slight conicity, imposed by molding production requirements.

In other words, the coupling appendage 22 may be comprised of a cylindrical or slightly conical body for the aforesaid reasons. Preferably, the maximum conicity of the coupling appendage 22 is 1/1.

Preferably, the head end 222 is counter-shaped with respect to the shoulder 113 in order to improve the fit between the head end and shoulder and thus ensure a better fit.

Advantageously, the cap 20 may be of the type to be engaged by screwing onto the container body 10 at the main gripping body 21.

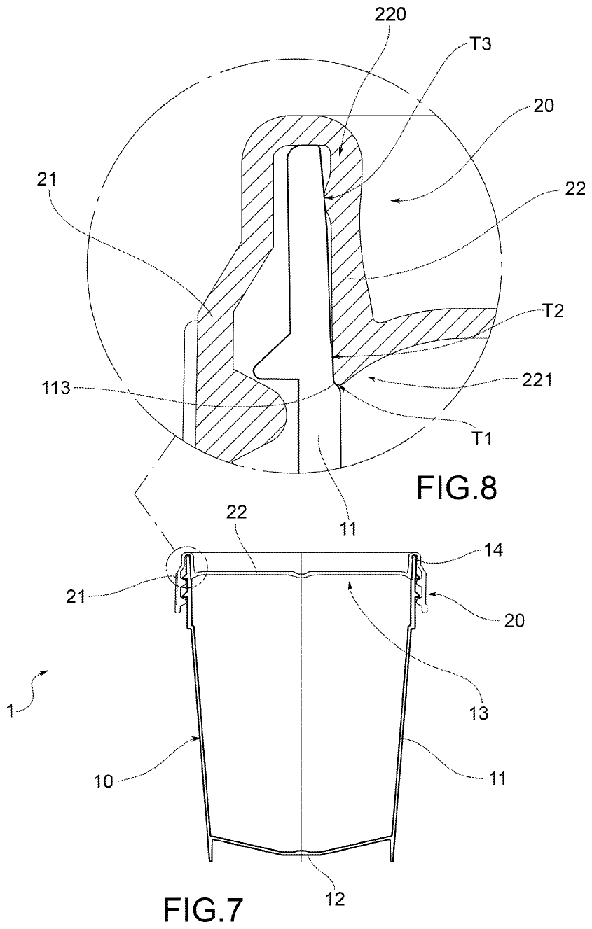

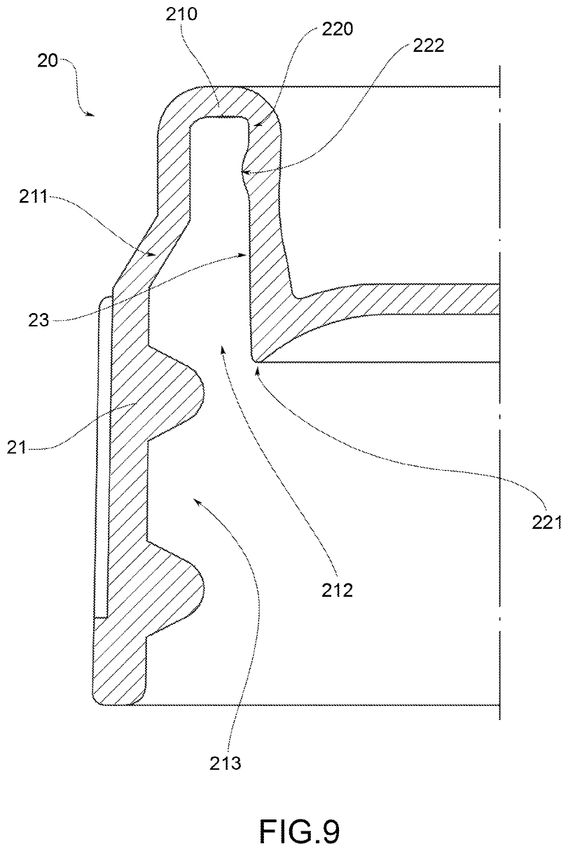

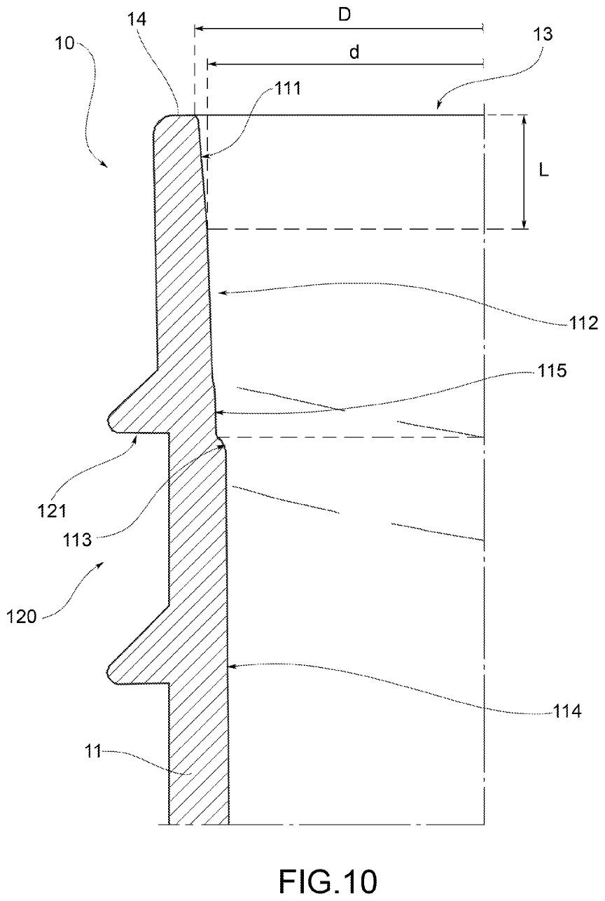

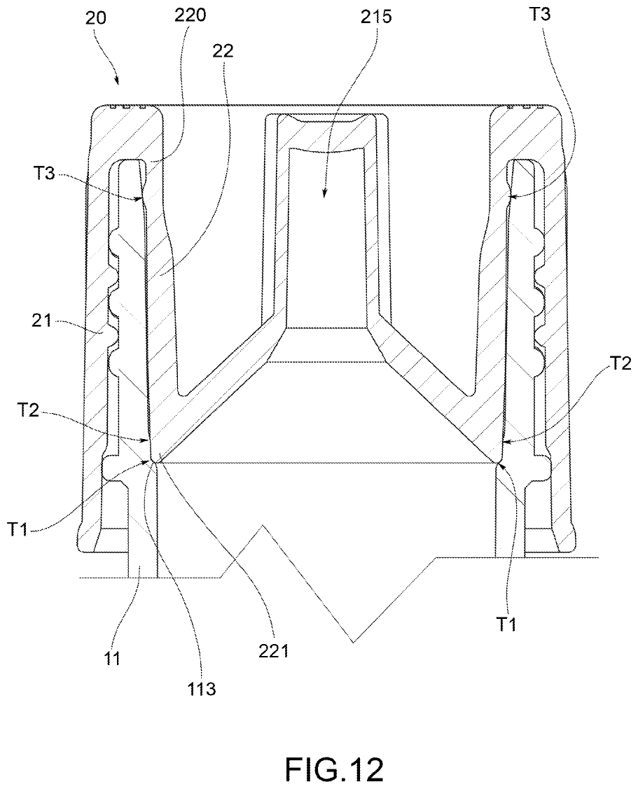

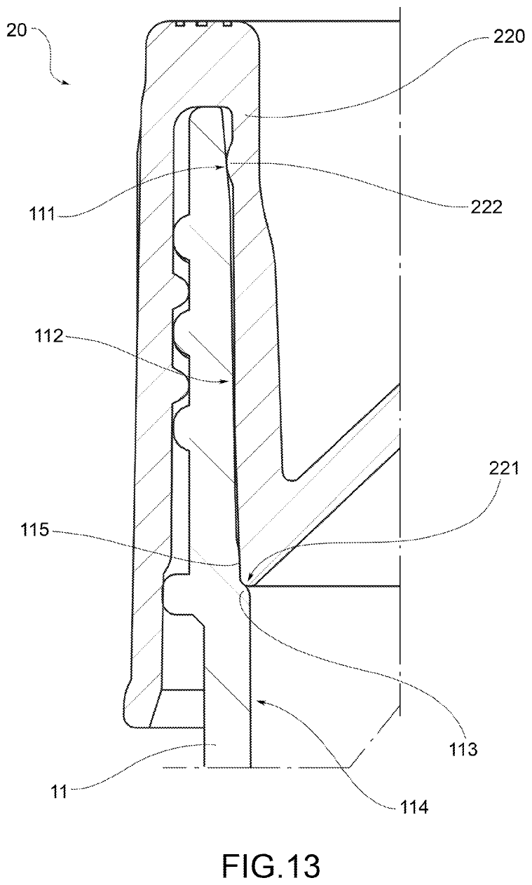

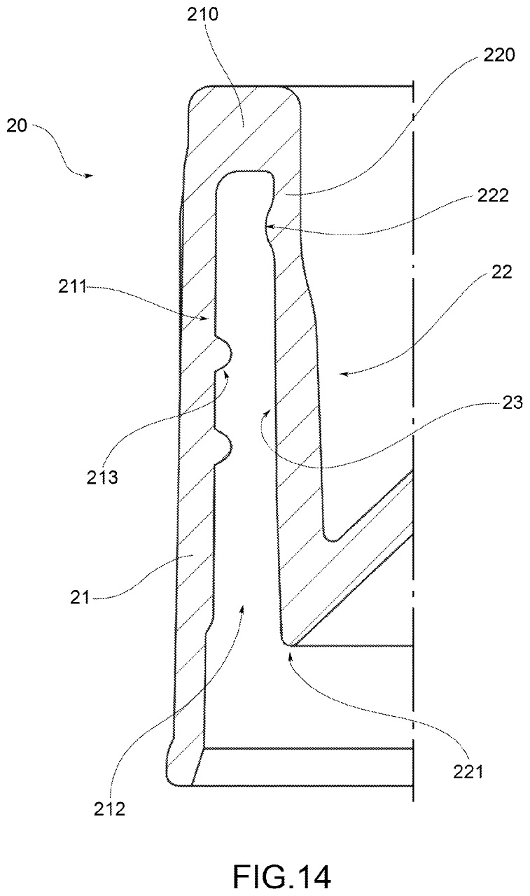

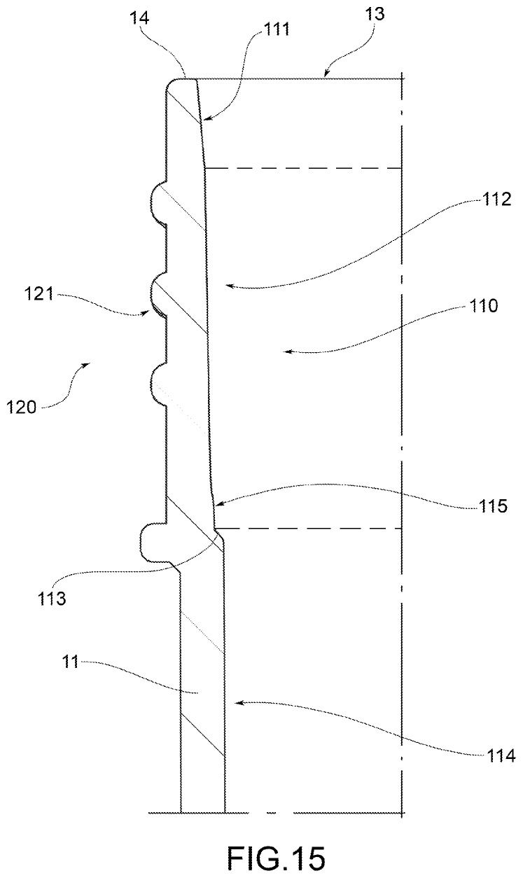

Specifically, as shown in the two embodiments of FIGS. 7 to 10 and 11 to 15 respectively, the main gripping body 21 is comprised of a cup-shaped body having a bottom 210 and a side wall 211. The coupling appendage 22 extends coaxially within the cup-shaped body from the bottom 210 such that an annular cavity 212 intended to partially accommodate therein a portion of said container body 10 is defined between the side wall 211 of said cup-shaped body and the coupling appendage. The side wall 211 comprises a threaded surface 213 that faces the inside of said cup-shaped body and is intended to engage in screwing with a counter-threaded surface 121 formed on the outer surface 120 of said container body 10.

Operatively, in such a case, the insertion of the coupling appendage 22 inside the container body 10 is guided and forced by screwing the cap onto the container body.

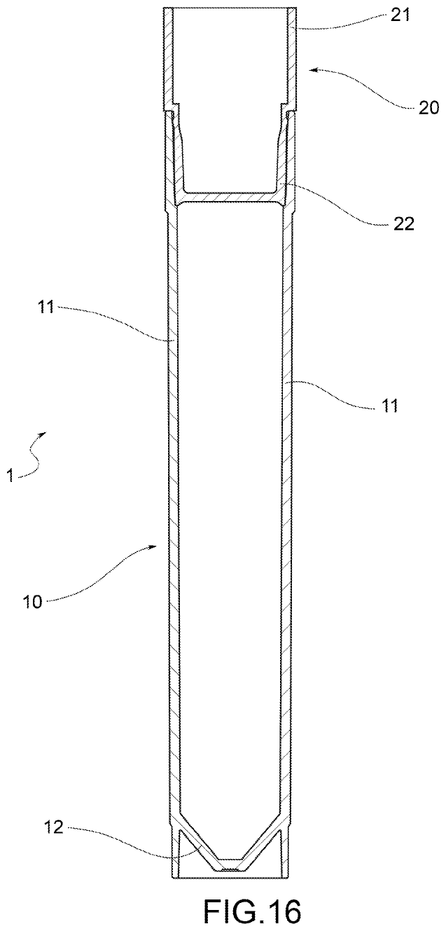

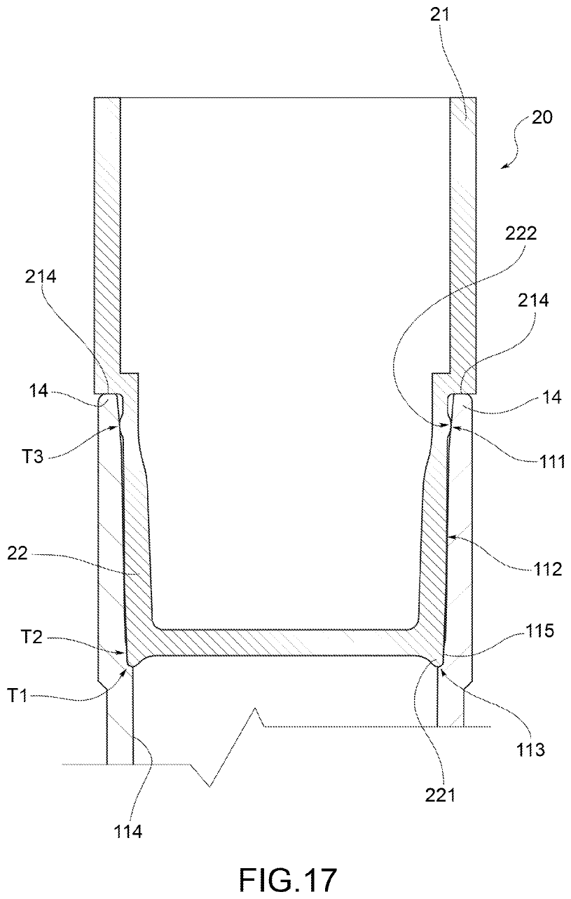

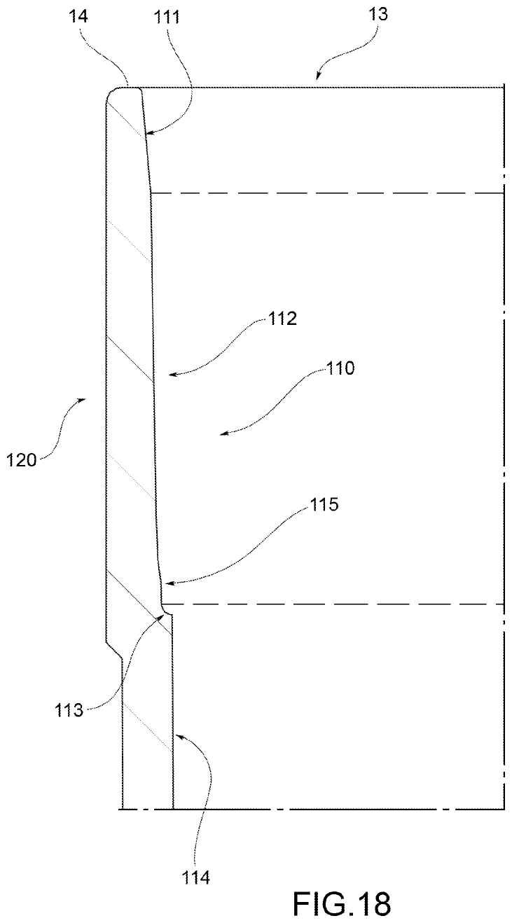

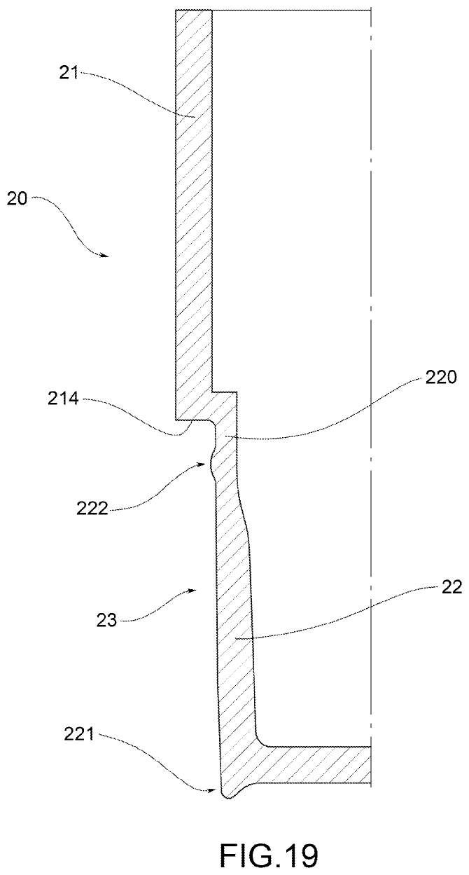

Alternatively, as shown in the embodiment of FIGS. 16 to 19, the cap 20 may also not be engageable by screwing onto the container body 10, but rather be a cap that is simply insertable by pressure, possibly by applying a relative rotational motion between the cap and the container body.

Specifically, as shown in FIGS. 16 to 19, the aforesaid main gripping body 21 defines, at the base 220 of the coupling appendage 22, an abutment ring 214 for the free edge 14 of said container body 10. The main gripping body 21 is configured to engage said container body 10 only at said abutment ring 214.

The hermetically sealable container 1 for liquids according to the invention may be used for the containment of any liquid.

Specifically, the container according to the invention is intended for the containment of liquids for which the risk of conveying the contents from the container to the external environment is to be reduced by means of the sealing cap.

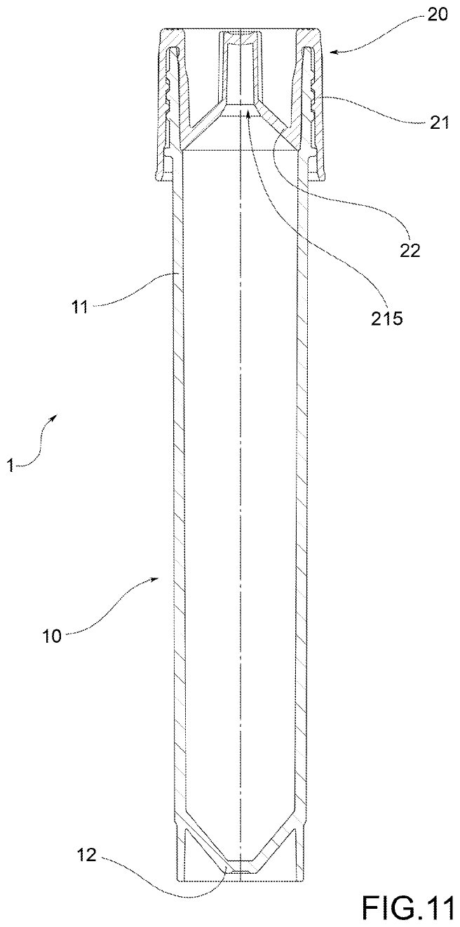

Still more specifically, the container according to the invention is intended for the containment of contaminating and/or hazardous liquids, such as biological samples. In this case, advantageously, the container may be made, in particular, in the form of a jar (as shown in FIGS. 7 to 10) or a test tube (as shown in FIGS. 11 to 15 and FIGS. 16 to 19).

In particular, the container 1 may be made in the form of a swab tube, in which, as shown in FIGS. 11 to 15, the coupling appendage 22 may be fitted with a seat 215, intended to be engaged by the support rod of a swab (not shown in the figures).

The invention allows numerous advantages to be obtained which have been explained throughout the description.

The hermetically sealable container 1 for liquids according to the invention, while being made entirely of plastics, significantly reduces the risk of contamination by conveying the contents to the external environment via the container cap.

The hermetically sealable container 1 for liquids according to the invention further provides a hermetic seal comparable to similar containers of known types.

Finally, the hermetically sealable container 1 for liquids according to the invention is simple and inexpensive to make, as it may be made in particular by the injection molding of plastics material.

The invention thus conceived therefore achieves its intended objects.

Obviously, in practice it may also assume different forms and configurations from the one illustrated above, without thereby departing from the present scope of protection.

Furthermore, all details may be replaced with technically equivalent elements, and the dimensions, shapes, and materials used may be any according to the needs.

1. A hermetically sealable container for liquids comprising:
a container body, delimiting an internal volume of the hermetically sealable container laterally by a tubular container wall and underneath by a container bottom, from which said tubular container wall extends, said container body being provided with a mouth delimited by a free edge of said tubular container wall; and
a cap, configured to close the mouth of the container body and make a hermetic seal therewith,
wherein said cap comprises a main gripping body, which is intended to remain outside said container body, and a coupling appendage, which is intended to be inserted inside said container body through said mouth, extends axially between a base and a head end, and comprises a lateral coupling surface intended to face an inner surface of said tubular container wall,
wherein the inner surface of said tubular container wall sequentially comprises, starting from the free edge towards the container bottom, a conical guide surface converging towards the container bottom, an intermediate surface connecting said conical guide surface to a shoulder, and an end surface connecting said shoulder to the container bottom, said intermediate surface defining a first annular prominence in proximity of said shoulder,
wherein the coupling appendage of said cap has an axial extension, so that the coupling appendage may be inserted inside the container body until the coupling appendage reaches said shoulder with its head end, and has a radial extension at the head end, so that, once the coupling appendage has reached the shoulder, the head end abuts against said shoulder to create a first sealing area between the cap and container body,
wherein the head end of the coupling appendage is radially dimensioned such that, when the head end abuts on said shoulder, the head end interferes with said first annular prominence, creating a second sealing zone between the cap and container body, and
wherein the coupling appendage of said cap comprises a second annular prominence that projects from said lateral coupling surface in proximity of the base of said coupling appendage and is axially positioned so that, when the head end abuts on said shoulder, said second annular prominence interferes with the conical guide surface, creating a third sealing zone between the cap and container body.

2. The hermetically sealable container of claim 1, wherein said conical guide surface has a conicity between 1/1 and 1/50.

3. The hermetically sealable container of claim 1, wherein said intermediate surface is conical converging towards the container bottom.

4. The hermetically sealable container of claim 3, wherein said intermediate surface has a conicity less than a conicity of the conical guide surface.

5. The hermetically sealable container of claim 1, wherein said intermediate surface is cylindrical.

6. The hermetically sealable container of claim 1, wherein said intermediate surface defines the first annular prominence close to said shoulder.

7. The hermetically sealable container of claim 1, wherein the lateral coupling surface of said coupling appendage is substantially cylindrical.

8. The hermetically sealable container of claim 1, wherein the head end is counter-shaped to said shoulder.

9. The hermetically sealable container of claim 1, wherein said cap is engageable by screwing onto said container body at said main gripping body.

10. The hermetically sealable container of claim 9, wherein said main gripping body is constituted by a cup-shaped body comprising a bottom and a side wall, wherein said coupling appendage extends coaxially inside said cup-shaped body starting from the bottom in such a way that, between the side wall of said cup-shaped body and said coupling appendage, an annular cavity is defined, the annular cavity being intended to partially accommodate a portion of said container body, and wherein the side wall comprises a threaded surface facing towards inside of said cup-shaped body and intended to engage by screwing a counter-threaded surface made on an outer surface of said container body.

11. The hermetically sealable container of claim 1, wherein said main gripping body defines at the base of said coupling appendage an abutment ring for the free edge of said container body, and wherein said main gripping body is configured to engage said container body only at said abutment ring.

12. The hermetically sealable container of claim 1, wherein said container body is made of a plastic material selected from the group consisting of polypropylene, polyethylene, polystyrene, polyethylene terephthalate (PET), polyvinyl chloride (PVC), polycarbonate, methacrylate and copolymers thereof.

13. The hermetically sealable container of claim 1, wherein said cap is made of a plastic material selected from the group consisting of polypropylene, polyethylene, polystyrene, polyethylene terephthalate (PET), polyvinyl chloride (PVC), polycarbonate, methacrylate and copolymers thereof.

14. The hermetically sealable container of claim 1, wherein said hermetically sealable container is a jar, in particular for containing biological samples.

15. The hermetically sealable container of claim 1, wherein said hermetically sealable container is a test tube, in particular for containing biological samples.
